(19) United States
(12) Patent Application Publication
(10) Pub. No.: US 2026/0257930 A1
(43) Pub. Date: Sep. 3, 2026

(54) METHOD FOR THE COMBINED REMOVAL OF HYDROGEN SULFIDE FROM A RAW GAS MIXTURE AMMONIA FROM A WASTE LIQUID

(71) Applicant: Bonno KOERS, DOESBURG (NL)

(72) Inventor: Bonno KOERS, DOESBURG (NL)

(21) Appl. No.: 19/489,474

(22) PCT Filed: May 31, 2024
(86) PCT No.: PCT/NL2024/050277

§ 371 Date: Dec. 2, 2025

(30) Foreign Application Priority Data
Jun. 2, 2023 (NL) 2034980

(51) Int. Cl. C01C 1/247; B01D 53/52; B01D 53/78

(52) U.S. Cl. CPC C01C 1/247; B01D 53/52; B01D 53/78; B01D 2251/2062; B01D 2257/304; B01D 2258/05

(57) ABSTRACT

The present disclosure is based on the combined removal of hydrogen sulfide from a raw gas mixture and removal of ammonia from a waste liquid. The disclosure relates to a method for the combined removal of hydrogen sulfide from a raw gas mixture and removal of ammonia from a waste liquid. The disclosure also relates to a method for the production of ammonium sulfate.

The present invention relates to the combined removal of hydrogen sulfide from a raw gas mixture and ammonia from a waste liquid which can be applied for the production of ammonium sulfate fertilizer.

### INTRODUCTION

Waste gas mixtures produced and used in industry may contain harmful sulfurous compounds such as hydrogen sulfide (H₂S). These sulfur compounds are very harmful to the environment and human health. In general they generate an unpleasant smell and even at very low concentrations it can be life threatening.

There is a continuous need in industry for improved methods and apparatuses which remove harmful H₂S from gas mixtures.

Also waste liquids containing high concentrations of ammonia (NH₃) may cause problems, because ammonia is a major atmospheric pollutant.

In particular in livestock agriculture, gas containing H₂S and manure with high concentrations of NH₃ are produced, which may both cause an environmental burden.

On the other hand, raw gas mixtures may contain valuable components (such as methane), but require removal of H₂S to allow further us of the gas to exploit the presence of these valuable components.

In this sense it is desirable to remove both the H₂S from raw gas mixtures as well as NH₃ from waste liquids.

### DESCRIPTION OF THE INVENTION

The present inventor has now found an efficient way to combine the removal of hydrogen sulfide from a raw gas mixture and ammonia from an ammonium containing liquid, which results in the production of ammonium sulfate fertilizer. On the other hand, H₂S is cleaned out of raw gas with the intent to allow the gas to be used in the grid or in combustion engines for energy.

The present invention relates in a first aspect to a method for the combined removal of hydrogen sulfide from a raw gas mixture and removal of ammonia from a waste liquid, comprising: contacting a stream of a raw gas mixture containing hydrogen sulfide and a stream of an alkaline aqueous slurry comprising ammonia in a first unit, wherein upon contacting said raw gas mixture with said slurry at least part of the hydrogen sulfide is dissolved into said slurry and at least partially converted to ammonium sulfide; passing said slurry from said first unit to a second unit wherein said slurry is subjected to aerobic conditions so as to effect oxidation of said ammonium sulfide to produce ammonium sulfate.

Ammonium sulfate ((NH₄)₂SO₄) is very suitable as a fertilizer for alkaline soils. In the soil the ammonium ion is released and forms a small amount of acid, lowering the pH balance of the soil, while contributing essential nitrogen for plant growth. Apart from that the salt is also a source of sulfur which is also a valuable crop nutrient.

Other uses of ammonium sulfate include its use as a food additive, in treatment of drinking water, as a flame retardant, etc. Common processes of production of ammonium sulfate include isolation of ammonium sulfate as a by-product from the production of caprolactam; synthetic preparation of ammonium sulfate by reacting anhydrous ammonia and sulfuric acid; and production of ammonium sulfate as a by-product of coke ovens, by reacting ammonia from coke oven gas with sulfuric acid.

In this respect the present invention does not only provide an effective approach to remove hydrogen sulfide from a raw gas mixture and ammonia from a waste liquid in one combined approach, it also results in a the production of a valuable product, namely ammonium sulfate.

Therefore the invention relates in a second aspect to a method for the production of ammonium sulfate, in particular an ammonium sulfate (organic) fertilizer, comprising: contacting a stream of a raw gas mixture containing hydrogen sulfide and a stream of an alkaline aqueous slurry comprising ammonia in a first unit, wherein upon contacting said raw gas mixture with said slurry at least part of the hydrogen sulfide is dissolved into said slurry and at least partially converted to ammonium sulfide; passing said slurry from said first unit to a second unit wherein said slurry is subjected to aerobic conditions so as to effect oxidation of said ammonium sulfide to produce ammonium sulfate.

In light of this, the method of the present invention allows simultaneous removal of hydrogen sulfide from a raw gas mixture and ammonia from an ammonium containing liquid, combined with the production of ammonium sulfate.

In order to make the ammonium sulfate suitable for further use, the method may further comprise drying the slurry after being subjected to said aerobic conditions to produce solid ammonium sulfate salt.

The aqueous slurry comprising ammonia in the first unit should be sufficiently alkaline in order to allow the H₂S from the raw gas mixture to dissolve in the aqueous slurry. For this purpose, the stream of an alkaline aqueous slurry comprising ammonia suitably has a pH of 8.5 or more, such as 8.5-11. It is preferred in this respect that the pH of the alkaline aqueous slurry comprising ammonia is in the range of 9-10.

It is preferred that contacting said stream of a raw gas mixture containing hydrogen sulfide and said stream of an alkaline aqueous slurry comprising ammonia in said first unit takes place under anaerobic conditions. This is important for the use of the cleaned gas later on. The ammonium created alkaline conditions ensure optimal uptake of H₂S under these anaerobic conditions after which it can be fully oxidized under aerobic conditions to ammonium sulfate in the second unit. The separation of the method according to the invention in an anaerobic and subsequent aerobic stage therefore ensures optimal H2S uptake, while increasing the economic value of the gas and full conversion to ammonium sulfate.

In addition, gas mixtures produced in industrial or agricultural environments often contain inflammable material, so that the presence of oxygen in the initial stage should be prevented or at least kept to a minimum, so as to avoid risks of fire and/or explosion. For instance, the raw gas mixture in the context may be selected from the group comprising boiler gas, landfill gas, flare gas and biogas, which all may contain an amount of methane (CH₄), which is highly inflammable. Also for this reason, it is preferred that contacting said stream of a raw gas mixture containing hydrogen sulfide and said stream of an alkaline aqueous slurry comprising ammonia in said first unit takes place under anaerobic conditions. While the H₂S dissolves in the aqueous medium, the methane remains in gaseous form and can as such be easily separated and discharged from the first unit in the form of a product gas containing a very low level of harmful sulfurous compounds (if still present at all) and a substantial level of methane. This methane can be combusted or oxidized with oxygen to release energy, for example for use in gas engines. In this respect it is preferred that in the first unit, the raw gas mixture containing methane and H₂S is contacted with the ammonia containing slurry by (optionally intermittently) spraying the gas mixture with said slurry in order to separate the methane and dissolve the H₂S in the slurry. This process, which is a chemical and in principle non-biological process, may suitably be carried out using any chemical scrubber, for instance a trickling filter, for example as disclosed in the present applicant's previous application EP 3 395 429 A1, the contents of which are incorporated herein by reference.

Anaerobic digestion of a biodegradable feedstock of organic matter produces digestate and biogas. This digestate is separated in a solid and an aqueous fraction. The solid fraction of the digestate can be separated from the liquid fraction by mechanical separation systems, like screw press, decanter centrifuge or dewatering drum. The solid fraction can be used for further purposes, for instance as a fertilizer or any other suitable purpose. The aqueous fraction on its turn usually contains high contents of ammonia (in equilibrium with ammonium—NH₄) and may also contain amounts of fine solids. On the other hand, the biogas produced in anaerobic digestion usually contains hydrogen sulfide at levels that above the acceptable levels for release in the environment. The present invention is therefore very suitable for processing the digestate and biogas produced by anaerobic digestion, because it allows to clean both the raw gas products of industrial or agricultural processes as well as their liquid waste streams in a combined process that can conveniently be carried out onsite. In line with this, the first unit in the context of the invention may be provided downstream of a digester that produces biogas from organic matter. This digester may also produce the abovementioned digestate that may be processed in accordance with the method of the invention.

It is of course also possible to combine a raw gas mixture from one source with a liquid ammonia containing waste product from another source.

In one embodiment, the invention finds application in the combined desulfurization of gas emissions and cleaning of the leachate of landfill.

As mentioned above, in livestock agriculture gas mixtures containing H₂S and manure digestate with high concentrations of NH₃ are produced. The aqueous fraction of the manure digestate may be separated from the solid fraction to result in an aqueous slurry that contains a high amount of ammonia. This makes the principle of the present invention very applicable in this technical area, because it allows to clean both the gaseous mixtures as well as the liquid waste streams onsite in a combined process. In accordance, in a preferred embodiment of the invention said alkaline aqueous slurry comprising ammonia is derived from a digestate from organic matter, such as manure.

It is noted that the liquid waste product of separating digestate is usually more or less at neutral pH, i.e. with a pH around 7 or lower. In order to allow suitable uptake of hydrogen sulfide from the gas mixture, the pH should therefore be adjusted to alkaline levels. Although this could in principle be done by adding caustic agents, the present inventor has found that the pH can be brought to suitable levels of 8.5-11 by removing carbon dioxide from the aqueous fraction of the digestate. Normally after evaporation of the carbon dioxide, the pH is between 9 and 10, because above pH 10 carbon dioxide evaporation takes place less efficiently. In this respect a preferred pH range is 9-10. In this respect it is preferred, if digestate is the source of the liquid waste product, that the method of the invention contains a step of removal of carbon dioxide from said digestate to produce said alkaline aqueous slurry comprising ammonia. The inventor has found that this can be done without the need for elaborated technical equipment by exposing said digestate or liquid fraction thereof to air because the partial pressure of carbon dioxide in the waste liquid is such high that it evaporates into the environment. To allow evaporation of carbon dioxide, the liquid fraction may initially be stored in a tank where carbon dioxide is evaporated until normally a more or less stable pH of up to 10 is achieved, followed by an optional filter step to remove any solids present in the liquid fraction, after which the liquid fraction is introduced into the first unit as aqueous slurry containing ammonia.

Once the hydrogen sulfide is dissolved in the aqueous slurry a chemical reaction takes place in the first unit wherein ammonium sulfide is produced from hydrogen sulfide and ammonia. In principle, this results in removal substantially all hydrogen sulfide from the raw gas mixture. Because ammonia is present in excess, allowing an efficient reaction and full conversion of hydrogen sulfide to take place, in general in this step the ammonia will be removed only partially from the waste liquid, while all or substantially all of the hydrogen sulfide is removed from the raw gas mixture.

Once the hydrogen sulfide is dissolved in the aqueous slurry, converted to ammonium sulfide and the optional inflammable compounds such as methane have been removed, the slurry can be passed to a second unit where under aerobic conditions oxidation of said ammonium sulfide takes place. This process produces ammonium sulfate, which as mentioned above, can be further processed for further use, for instance as a fertilizer.

Where in the first unit the anaerobic reaction is a chemical reaction, the oxidation process in the second unit is in principle a biologically catalyzed reaction.

In this respect, the aerobic oxidation of ammonium sulfate and any remaining hydrogen sulfide is suitably effected by aerobic micro-organisms, such as bacteria mixtures of bacteria which are known in the art for purposes of removing harmful sulfurous compounds, such as H₂S, CS₂ and SOₓ from gases, liquids or fluids. Bacterial cultures for aerobic and/or anaerobic conversion of harmful sulfurous compounds are known and available in the art. Such bacteria may for example include bacteria belonging to the Thiobacillus genus. The slurry may optionally comprise additional food sources for the micro-organisms or other supplements. The microorganisms may be present in the slurry and/or as biofilms in the second unit. Also the second unit may be designed as a (bio)trickling filter. It is also possible to use an aerated water container, although this will normally be less efficient than a trickling filter.

By way of example, the method according to the present invention may in principle be carried out with an apparatus as disclosed in the present applicant's earlier application WO2016/204616 (A1), the contents of which are incorporated herein by reference, in particular in reference to FIGS. 1 and 2 thereof, albeit in principle without recycling the liquid stream from the second unit to the first unit.

1. A method for the combined removal of hydrogen sulfide from a raw gas mixture and removal of ammonia from a waste liquid, comprising:
contacting a stream of a raw gas mixture containing hydrogen sulfide and a stream of an alkaline aqueous slurry comprising ammonia in a first unit, wherein upon contacting said raw gas mixture with said slurry at least part of the hydrogen sulfide is dissolved into said slurry and at least partially converted to ammonium sulfide; and
passing said slurry from said first unit to a second unit wherein said slurry is subjected to aerobic conditions so as to effect oxidation of said ammonium sulfide to produce ammonium sulfate.

2. A method for the production of ammonium sulfate, comprising:
contacting a stream of a raw gas mixture containing hydrogen sulfide and a stream of an alkaline aqueous slurry comprising ammonia in a first unit, wherein upon contacting said raw gas mixture with said slurry at least part of the hydrogen sulfide is dissolved into said slurry and at least partially converted to ammonium sulfide; and
passing said slurry from said first unit to a second unit wherein said slurry is subjected to aerobic conditions so as to effect oxidation of said ammonium sulfide to produce ammonium sulfate.

3. The method according to claim 1, further comprising drying the slurry after being subjected to said aerobic conditions to produce solid ammonium sulfate salt.

4. The method according to claim 1, wherein said second stream of an alkaline aqueous slurry comprising ammonia has a pH of 8.5 to 11 or more.

5. The method according to claim 4, wherein said second stream of an alkaline aqueous slurry comprising ammonia has a pH of 9 to 10.

6. The method according to claim 1, wherein said raw gas mixture contains inflammable material, and wherein contacting said stream of a raw gas mixture containing hydrogen sulfide and said stream of an alkaline aqueous slurry comprising ammonia in said first unit takes place under anaerobic conditions.

7. The method according to claim 6, wherein the inflammable material comprises methane.

8. The method according to claim 1, wherein the raw gas mixture is selected from the group comprising boiler gas, landfill gas, flare gas and biogas.

9. The method according to claim 1, wherein said alkaline aqueous slurry comprising ammonia is derived from a digestate from organic matter and wherein the method contains a step of removal of carbon dioxide from said digestate or the liquid fraction thereof to produce said alkaline aqueous slurry comprising ammonia.

10. The method according to claim 9, wherein said digestate is a manure digestate.

11. The method according to claim 9, wherein said carbon dioxide is removed by exposing said digestate or liquid fraction thereof to air.

12. The method according to claim 11, wherein the raw gas mixture is biogas and wherein said first unit is provided downstream of a digester that produces biogas from organic matter.

13. The method according to claim 12, wherein said digester also produces a digestate, and wherein the method contains a step of removal of carbon dioxide from said digestate or liquid fraction thereof to produce said alkaline aqueous slurry comprising ammonia.

14. The method according to claim 13, wherein said digestate is a manure digestate.

15. The method according to claim 1, wherein said first unit is a trickling filter and/or wherein said second unit is a trickling filter, in particular wherein the second unit is a biotrickling filter.

16. The method according to claim 2, further comprising drying the slurry after being subjected to said aerobic conditions to produce solid ammonium sulfate salt.

17. The method according to claim 2, wherein said second stream of an alkaline aqueous slurry comprising ammonia has a pH of 8.5 to 11 or more.

18. The method according to claim 17, wherein said second stream of an alkaline aqueous slurry comprising ammonia has a pH of 9 to 10.

19. The method according to claim 2, wherein said raw gas mixture contains inflammable material, and wherein contacting said stream of a raw gas mixture containing hydrogen sulfide and said stream of an alkaline aqueous slurry comprising ammonia in said first unit takes place under anaerobic conditions.

20. The method according to claim 19, wherein the inflammable material comprises methane.

21. The method according to claim 2, wherein the raw gas mixture is selected from the group comprising boiler gas, landfill gas, flare gas and biogas.

22. The method according to claim 2, wherein said alkaline aqueous slurry comprising ammonia is derived from a digestate from organic matter and wherein the method contains a step of removal of carbon dioxide from said digestate or the liquid fraction thereof to produce said alkaline aqueous slurry comprising ammonia.

23. The method according to claim 22, wherein said digestate is a manure digestate.

24. The method according to claim 22, wherein said carbon dioxide is removed by exposing said digestate or liquid fraction thereof to air.

25. The method according to claim 24, wherein the raw gas mixture is biogas and wherein said first unit is provided downstream of a digester that produces biogas from organic matter.

26. The method according to claim 25, wherein said digester also produces a digestate, and wherein the method contains a step of removal of carbon dioxide from said digestate or liquid fraction thereof to produce said alkaline aqueous slurry comprising ammonia.

27. The method according to claim 21, wherein said digestate is a manure digestate.

28. The method according to claim 2, wherein said first unit is a trickling filter and/or wherein said second unit is a trickling filter, in particular wherein the second unit is a biotrickling filter.
